(19) United States
(12) Patent Application Publication
(10) Pub. No.: US 2026/0306557 A1
(43) Pub. Date: Oct. 8, 2026

(54) COMPOSITION INCLUDING PROPIONIBACTERIUM FREUDENREICHII MJ2 STRAINDERIVED EXTRACELLULAR VESICLES OR CHAPERONIN PROTEIN AS ACTIVE INGREDIENT FOR PREVENTION, ALLEVIATION, OR TREATMENT OF RHEUMATOID ARTHRITIS

(71) Applicant: KOREA UNIVERSITY RESEARCH AND BUSINESS FOUNDATION, Seoul (KR)

(72) Inventors: Young Hee LIM, Seoul (KR); Joo Young CHO, Gunsan-si (KR); Hee Eun WOO, Seoul (KR); Ji Ah YEOM, Seoul (KR)

(73) Assignee: KOREA UNIVERSITY RESEARCH AND BUSINESS FOUNDATION, Seoul (KR)

(21) Appl. No.: 19/480,508

(22) PCT Filed: Nov. 1, 2023
(86) PCT No.: PCT/KR2023/017254

§ 371 Date: Oct. 31, 2025

(30) Foreign Application Priority Data
May 2, 2023 (KR) 10-2023-0057127

(51) Int. Cl. A61K 35/74; A61K 38/16; A61P 19/02; A61P 29/00

(52) U.S. Cl. CPC A61K 35/74; A61K 38/164; A61P 19/02; A61P 29/00

(57) ABSTRACT

The present invention relates to a composition including Propionibacterium freudenreichii MJ2 strain-derived extracellular vesicles or chaperonin protein as an active ingredient for the prevention, alleviation, or treatment of rheumatoid arthritis. Specifically, the present invention relates to a pharmaceutical composition for the prevention or treatment of rheumatoid arthritis and a food composition for the prevention or alleviation of rheumatoid arthritis, each containing, as an active ingredient, extracellular vesicles or 60 kDa chaperonin protein (Cpn60) derived from Propionibacterium freudenreichii strain.

### TECHNICAL FIELD

The present disclosure relates to a composition including a Propionibacterium freudenreichii MJ2 strain-derived extracellular vesicle or chaperonin protein as an active ingredient for the prevention, alleviation, or treatment of rheumatoid arthritis.

### BACKGROUND ART

Rheumatoid arthritis is a disease that occurs due to inflammation of the synovium, the tissue surrounding the joints, and it can develop in almost any movable joint and progress as a chronic disease over several months to years. Rheumatoid arthritis is caused by an abnormal immune system and can cause problems in various organs in addition to the joints, but the most typical early symptoms include swelling and pain in the joints of the hands and feet, and symptoms may also appear in the knees, shoulders, elbows, or temporomandibular joints. In addition, inflammation can occur in organs other than the joints, such as the heart and blood vessels, and it has been reported that interstitial lung disease may develop.

Osteoclasts are cells that destroy and resorb bone and are characterized by being multinucleated cells. Osteoclasts abnormally differentiate under inflammatory responses, being induced by the inflammation-inducing factor receptor activator of nuclear factor kappa-B ligand (RANKL), which binds to RANK, a receptor for RANKL on the surface of osteoclast precursor cells, thereby inducing differentiation into osteoclasts. Therefore, inhibition of osteoclast differentiation can serve as a target for preventing and treating arthritis, and substances that inhibit osteoclast differentiation can be therapeutic agents capable of preventing or treating arthritis.

Currently, therapeutic agents for rheumatoid arthritis include antirheumatic drugs that can prevent joint damage, nonsteroidal anti-inflammatory drugs (NSAIDS), and immunosuppressants. However, although NSAIDs have the effect of relieving pain, they do not suppress the course of the disease and can cause gastrointestinal disorders when taken for a long period. Immunosuppressants, such as TNF-α inhibitors, may cause side effects such as latent tuberculosis. Although the efficacy of current therapeutic agents has improved compared to the past, there are still problems of side effects, high cost, and insufficient therapeutic efficacy relative to long-term administration.

Therefore, there is a need to develop new therapeutic agents that more effectively suppress rheumatoid arthritis symptoms, have safety, and exhibit excellent therapeutic effects.

Meanwhile, extracellular vesicles (EVs) refer to membrane-bound vesicles secreted by cells. Extracellular vesicles are composed of a bilayer lipid membrane and contain nucleic acids, proteins, lipids, etc. In the past, extracellular vesicles were considered cellular waste products; however, several studies have reported that extracellular vesicles play important roles in intercellular communication, immune function, and diagnosis of specific diseases. Accordingly, extracellular vesicles have been developed and used as biomarkers for diseases, but research on using their intrinsic efficacy as therapeutic agents for specific diseases is still insufficient. Moreover, to date, there has been no report of isolating extracellular vesicles derived from probiotic microorganisms and using them as a therapeutic agent for rheumatoid arthritis.

Leading to the present disclosure, intensive and through research conducted by the present inventors with the aim of developing a novel therapeutic agent for rheumatoid arthritis, succeeded in isolating extracellular vesicles from Propionibacterium freudenreichii MJ2 strain, which had been previously isolated and identified by the inventors, and found that the isolated extracellular vesicles and the chaperonin proteins contained therein exhibited excellent inhibitory effects against osteoclast differentiation and, when administered to collagen-induced rheumatoid arthritis mice, effectively prevented, improved, and treated rheumatoid arthritis.

### DISCLOSURE OF INVENTION

### Technical Problem

Accordingly, an aspect of the present disclosure is to provide a pharmaceutical composition including Propionibacterium freudenreichii strain-derived extracellular vesicles or 60 kDa chaperonin protein (Cpn60) as an active ingredient for the prevention or treatment of rheumatoid arthritis.

Another aspect of the present disclosure is to provide a food composition including Propionibacterium freudenreichii strain-derived extracellular vesicles or 60 kDa chaperonin protein (Cpn60) as an active ingredient for the prevention or alleviation of rheumatoid arthritis.

### Solution to Problem

In order to achieve the above-described goals, the present disclosure provides a pharmaceutical composition including Propionibacterium freudenreichii strain-derived extracellular vesicles or 60 kDa chaperonin protein (Cpn60) as an active ingredient for the prevention or treatment of rheumatoid arthritis.

In an embodiment of the present disclosure, the Propionibacterium freudenreichii may be Propionibacterium freudenreichii MJ2 (accession number KCCM12272P) strain.

In an embodiment of the present disclosure, the composition may have an activity of inhibiting the expression of osteoclast differentiation-related factors; inhibiting production of inflammatory cytokines;

inhibiting tartrate-resistant acid phosphatase (TRAP) activity in osteoclasts; increasing expression of IL-10;

alleviating bone erosion; or inhibiting production of collagen antigen-specific IgG, IgG1, and IgG2a antibodies.

In an embodiment of the present disclosure, the extracellular vesicles may be contained in the composition in an amount of 1×10⁶ particles/mL to 1×10⁸ particles/mL.

In an embodiment of the present disclosure, the extracellular vesicles may have an average particle size of 150-200 nm.

The present disclosure also provides a food composition including Propionibacterium freudenreichii strain-derived extracellular vesicles or 60 kDa chaperonin protein (Cpn60) as an active ingredient for the prevention or alleviation of rheumatoid arthritis.

In an embodiment of the present disclosure, the Propionibacterium freudenreichii may be Propionibacterium freudenreichii MJ2 (accession number KCCM12272P) strain.

In an embodiment of the present disclosure, the composition may have an activity of inhibiting the expression of osteoclast differentiation-related factors; inhibiting production of inflammatory cytokines; inhibiting tartrate-resistant acid phosphatase (TRAP) activity in osteoclasts; increasing expression of IL-10; alleviating bone erosion; or inhibiting production of collagen antigen-specific IgG, IgG1, and IgG2a antibodies.

In an embodiment of the present disclosure, the extracellular vesicles may be contained in the composition in an amount of 1×10⁶ particles/mL to 1×10⁸ particles/mL.

In an embodiment of the present disclosure, the extracellular vesicles may have an average particle size of 150-200 nm.

### Advantageous Effects of Invention

The extracellular vesicles and 60 kDa chaperonin protein (Cpn60) isolated from Propionibacterium freudenreichii MJ2 strain according to the present disclosure can inhibit expression of osteoclast differentiation-related factors, suppress production of inflammatory cytokines, inhibit TRAP activity, increase IL-10 expression, alleviate bone erosion, and inhibit production of collagen antigen-specific IgG, IgG1, and IgG2a antibodies. In addition to having these activities, they do not cause cytotoxicity or hepatotoxicity, ensuring in vivo safety, and therefore can be advantageously used for manufacturing pharmaceuticals and health functional foods for preventing, alleviating, or treating rheumatoid arthritis through regulation of excessive immune and inflammatory responses.

### BRIEF DESCRIPTION OF DRAWINGS

FIG. 1 shows a photograph of the extracellular vesicles isolated from Propionibacterium freudenreichii MJ2 strain of the present disclosure, observed using a scanning electron microscope (SEM).

FIG. 2 shows a photograph of the extracellular vesicles isolated from Propionibacterium freudenreichii MJ2 strain of the present disclosure, observed using a transmission electron microscope (TEM).

FIG. 3 shows the results of measuring the extracellular vesicles isolated from Propionibacterium freudenreichii MJ2 strain of the present disclosure by nanoparticle tracking analysis (NTA).

FIG. 4 shows the results of analyzing the cytotoxicity of the extracellular vesicles isolated from Propionibacterium freudenreichii MJ2 strain of the present disclosure.

FIG. 5 shows the results of analyzing the osteoclast differentiation inhibitory activity of the extracellular vesicles isolated from Propionibacterium freudenreichii MJ2 strain of the present disclosure.

FIG. 6 shows the results of confirming the inhibitory activity of the extracellular vesicles isolated from Propionibacterium freudenreichii MJ2 strain of the present disclosure on the expression of osteoclast differentiation-inducing factor genes.

FIG. 7 shows the results of confirming the inhibitory activity of the extracellular vesicles isolated from Propionibacterium freudenreichii MJ2 strain of the present disclosure on the expression of osteoclast differentiation-inducing factor proteins.

FIG. 8 shows the results of confirming the efficacy of improving arthritis in a collagen-induced arthritis mouse model by the extracellular vesicles isolated from Propionibacterium freudenreichii MJ2 strain of the present disclosure.

FIG. 9 shows the results of measuring the levels of hepatotoxicity indicators in a collagen-induced arthritis mouse model administered with the extracellular vesicles isolated from Propionibacterium freudenreichii MJ2 strain of the present disclosure.

FIG. 10 shows the results of confirming the efficacy of improving knee joint damage in a collagen-induced arthritis mouse model by the extracellular vesicles isolated from Propionibacterium freudenreichii MJ2 strain of the present disclosure.

FIG. 11 shows the results of confirming the effect of reducing collagen antigen-specific antibody concentrations in a collagen-induced arthritis mouse model by the extracellular vesicles isolated from Propionibacterium freudenreichii MJ2 strain of the present disclosure.

FIG. 12 shows the results of confirming the effects of decreasing expression of inflammatory cytokines and increasing expression of anti-inflammatory cytokines in a collagen-induced arthritis model mouse by the extracellular vesicles isolated from Propionibacterium freudenreichii MJ2 strain of the present disclosure.

FIG. 13 shows the results of confirming the inhibition of gene expression of osteoclast differentiation-inducing factors and the increase in gene expression of osteoclast differentiation-inhibitory factors in a collagen-induced arthritis mouse model by the extracellular vesicles isolated from Propionibacterium freudenreichii MJ2 strain of the present disclosure.

FIG. 14 shows the results of confirming the osteoclast differentiation inhibitory activity in a collagen-induced arthritis mouse model by the extracellular vesicles isolated from Propionibacterium freudenreichii MJ2 strain of the present disclosure.

FIG. 15 shows the results of synthesizing, isolating, and purifying the chaperonin protein derived from Propionibacterium freudenreichii MJ2 strain of the present disclosure.

FIG. 16 shows the results of analyzing the cytotoxicity of the chaperonin protein derived from Propionibacterium freudenreichii MJ2 strain of the present disclosure.

FIG. 17 shows the results of confirming the osteoclast differentiation inhibitory activity of the chaperonin protein derived from Propionibacterium freudenreichii MJ2 strain of the present disclosure.

FIG. 18 shows the results of confirming the inhibitory activity on the expression of osteoclast differentiation-inducing factor genes by the chaperonin protein derived from Propionibacterium freudenreichii MJ2 strain of the present disclosure.

FIG. 19 shows the results of confirming the efficacy of improving arthritis in a collagen-induced arthritis mouse model by the chaperonin protein derived from Propionibacterium freudenreichii MJ2 strain of the present disclosure.

FIG. 20 shows the results of measuring the levels of hepatotoxicity indicators in a collagen-induced arthritis mouse model administered with the chaperonin protein derived from Propionibacterium freudenreichii MJ2 strain of the present disclosure.

FIG. 21 shows the results of confirming the bone erosion improvement effect in a collagen-induced arthritis mouse model by the chaperonin protein derived from Propionibacterium freudenreichii MJ2 strain of the present disclosure.

FIG. 22 shows the results of confirming the effect of reducing collagen antigen-specific antibody concentrations in a collagen-induced arthritis mouse model by the chaperonin protein derived from Propionibacterium freudenreichii MJ2 strain of the present disclosure.

FIG. 23 shows the results of confirming the effect of reducing the expression of inflammatory cytokines in a collagen-induced arthritis mouse model by the chaperonin protein derived from Propionibacterium freudenreichii MJ2 strain of the present disclosure.

FIG. 24 shows the results of confirming the inhibitory activity on the expression of osteoclast differentiation-inducing factor genes in a collagen-induced arthritis mouse model by the chaperonin protein derived from Propionibacterium freudenreichii MJ2 strain of the present disclosure.

FIG. 25 shows the results of confirming the osteoclast differentiation inhibitory activity in a collagen-induced arthritis mouse model by the chaperonin protein derived from Propionibacterium freudenreichii MJ2 strain of the present disclosure.

### BEST MODE FOR CARRYING OUT THE INVENTION

In the present disclosure aiming to develop a novel therapeutic agent capable of effectively alleviating or treating rheumatoid arthritis, extracellular vesicles or 60 kDa chaperonin protein (Cpn60) derived from Propionibacterium freudenreichii MJ2 strain, which was isolated and identified by the inventors from raw milk, first found to have an activity of effectively was preventing, alleviating, or treating rheumatoid arthritis.

Accordingly, the present disclosure is characterized by providing a pharmaceutical composition including extracellular vesicles or 60 kDa chaperonin protein (Cpn60) derived from Propionibacterium freudenreichii strain as an active ingredient for preventing or treating rheumatoid arthritis.

Specifically, the Propionibacterium freudenreichii strain may be Propionibacterium freudenreichii MJ2 (accession number KCCM12272P) strain.

In the present disclosure, Propionibacterium freudenreichii MJ2 strain was first isolated by the inventors by confirming the presence of P. freudenreichii in raw milk obtained from a farm in Gimpo, Gyeonggi-do using the next generation sequencing (NGS) method, culturing same in a YELA medium for 7 days at 30° C. under anaerobic conditions, picking single colonies to select candidate strains, and performing 16s rDNA sequencing (universal primer: 1492R). The strain was then deposited with the Korean Culture Center of Microorganisms (KCCM) on Jun. 8, 2018, and was assigned the accession number KCCM 12272P.

In order to identify the pharmacologically active component of Propionibacterium freudenreichii MJ2 strain having a therapeutic effect on rheumatoid arthritis, the strain itself (live cells, killed cells), the culture (culture including the strain), the culture supernatant (culture medium with the strain removed), and extracellular vesicles isolated from the culture supernatant were analyzed for the degree of inhibition of osteoclast differentiation.

As a result, it was found that the extracellular vesicles isolated from Propionibacterium freudenreichii MJ2 strain exhibited a remarkably superior inhibitory effect against osteoclast differentiation compared to the strain itself (live cells, killed cells) and that this effect was also superior to those of the culture (including the strain) and the culture supernatant (with the strain removed).

Accordingly, the following experiments were performed to determine whether the extracellular vesicles isolated from Propionibacterium freudenreichii MJ2 strain could alleviate, prevent, or treat rheumatoid arthritis.

In an embodiment the 41 of present disclosure, Propionibacterium freudenreichii MJ2 strain was cultured at 30° C. for 48 hours, and the culture medium was centrifuged at 10,000×g for 20 minutes at 4° C. to obtain the supernatant. The extracellular vesicles larger than 100 kDa were using separated a filter, and then centrifuged again at 150,000×g for 3 hours at 4° C. to obtain the extracellular vesicles isolated from the strain.

The extracellular vesicles obtained by the above method were then analyzed for characteristics. The isolated extracellular vesicles of the present disclosure were measured to have a particle size of about 150-200 nm and preferably about 170 nm, with a spherical shape.

In another embodiment of the present disclosure, proteomic analysis was performed on the proteins contained in the isolated extracellular vesicles, discovering that 20 types of proteins, including 60 kDa chaperonin, Chain A, and methylmalonyl-CoA carboxyltransferase 12S subunit protein, as shown in Table 1 below, were predominant, especially with the confirmation of prophylactic, palliative, and therapeutic effects of 60 kDa chaperonin protein on rheumatoid arthritis.

First, in order to confirm whether the extracellular vesicles isolated from Propionibacterium freudenreichii MJ2 strain inhibit osteoclast differentiation, a macrophage cell line was treated with receptor activator of nuclear factor kappa-B ligand (RANKL) to induce osteoclast differentiation, and then additionally with the extracellular vesicles of the present disclosure at various concentrations. Subsequently, the expression levels of osteoclast differentiation markers and the number of TRAP-activated osteoclasts were analyzed. As a result, it was confirmed that groups treated with 1×10⁶ particles/mL to 1×10⁸ particles/mL of extracellular vesicles were unlikely to undergo osteoclast differentiation compared with the untreated control group. Meanwhile, the group treated with 1×10⁹ particles/mL of extracellular vesicles was observed to undergo cell death.

In another embodiment of the present disclosure, the extracellular vesicles isolated from Propionibacterium freudenreichii MJ2 strain were administered to collagen-induced rheumatoid arthritis mice (the extracellular vesicles were administered at doses of 1×10⁷ particles/mL and 1×10⁸ particles/mL), and the improvement of the disease was observed. As a result, the symptoms of rheumatoid arthritis were visually observed to be alleviated in the group administered with the extracellular vesicles of the present disclosure. It was also confirmed that the extracellular vesicles alleviated bone erosion and knee joint damage and significantly reduced the concentrations of total IgG, IgG1, and IgG2a antibodies specific to the collagen antigen, which are elevated in the blood upon induction of rheumatoid arthritis.

Accordingly, these results indicate that the extracellular vesicles isolated from Propionibacterium freudenreichii MJ2 strain have the effect of preventing, alleviating, and treating rheumatoid arthritis and that a preferable effect can be obtained when using the extracellular vesicles of the present disclosure in an amount of 1×10⁶ particles/mL to 1×10⁸ particles/mL.

Furthermore, in another embodiment of the present disclosure, the 60 kDa chaperonin protein (Cpn60), which was identified as a major protein contained in the extracellular vesicles, was synthesized, and analyzed for inhibitory activity against osteoclast differentiation. As a result, it was found that the 60 kDa chaperonin protein (Cpn60) also has the effect of inhibiting osteoclast differentiation.

The 60 kDa chaperonin protein is a protein conserved through evolutionary stages and indispensable in both eukaryotic and prokaryotic cells. In addition to its intrinsic protein-folding function, the chaperonin protein is known to act as a receptor that binds ligands inside and outside the cell and as an intercellular signaling molecule; however, there has been no study to date reporting that the 60 kDa chaperonin protein can prevent, alleviate, and treat rheumatoid arthritis.

Furthermore, in order to confirm whether the extracellular vesicles and 60 kDa chaperonin protein isolated from Propionibacterium freudenreichii MJ2 strain actually have pharmacological efficacy for treating rheumatoid arthritis, rheumatoid arthritis was induced by administering collagen to mice to which the extracellular vesicles or 60 kDa chaperonin protein of the present disclosure had been administered, followed by analyzing the arthritis clinical scores of each mouse.

As a result, it was found that in the mice in which rheumatoid arthritis was induced without administration of extracellular vesicles or 60 kDa chaperonin protein, the arthritis clinical score increased over time and the symptoms worsened, whereas in the groups administered with the extracellular vesicles or 60 kDa chaperonin protein of the present disclosure, the arthritis clinical score was significantly reduced, indicating alleviation of rheumatoid arthritis symptoms.

These results confirmed that the extracellular vesicles or 60 kDa chaperonin protein of the present disclosure can prevent and alleviate rheumatoid arthritis.

In another embodiment, experiments were performed to examine whether the extracellular vesicles or 60 kDa chaperonin protein of the present disclosure can alleviate bone erosion, which is a symptom observed when rheumatoid arthritis develops, wherein cells causing inflammation erode the bone surface, leading to bone melting or deep pitting.

As a result, it was found that the group of mice administered with the extracellular vesicles or 60 kDa chaperonin protein of the present disclosure had a significantly reduced area of bone erosion, with the concomitant alleviation of knee joint damage, compared to the control group without administration.

In addition, in order to serologically evaluate the severity of autoimmune disease symptoms of rheumatoid arthritis, the amount of collagen antigen-specific IgG in plasma was measured, and it was found that in the plasma of disease control mice in which rheumatoid arthritis was induced, the production of collagen antigen-specific IgG antibodies increased, whereas in the group of mice administered with the extracellular vesicles or 60 kDa chaperonin protein of the present disclosure, the concentrations of total IgG, IgG1, and IgG2a antibodies were significantly reduced compared with the disease control group.

Moreover, it was found that the group of mice administered with the extracellular vesicles or 60 kDa chaperonin protein of the present disclosure exhibited significantly decreased production levels of inflammatory cytokines and an increased expression level of IL-10, an anti-inflammatory cytokine, compared with the disease control group.

These results indicate that the extracellular vesicles or 60 kDa chaperonin protein of the present disclosure can suppress excessive immune and inflammatory responses and that rheumatoid arthritis can be prevented, alleviated, and treated through regulation of immune and inflammatory responses.

Accordingly, the present disclosure may provide a pharmaceutical composition including extracellular vesicles or 60 kDa chaperonin protein (Cpn60) derived from Propionibacterium freudenreichii strain as an active ingredient for preventing or treating rheumatoid arthritis.

The pharmaceutical composition according to the present disclosure may further include a pharmaceutically acceptable carrier in addition to the active ingredient. The pharmaceutically acceptable carrier is one that is commonly used in formulation, and may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, but with no limitations thereto. In addition to these components, lubricants, wetting agents, sweeteners, flavoring agents, emulsifiers, suspending agents, and preservatives may further be included.

The pharmaceutical composition of the present disclosure may be administered orally or parenterally (for example, intravenously, subcutaneously, intraperitoneally, or topically) according to the intended method. The dosage may vary depending on the condition and weight of the patient, severity of the disease, dosage form, route of administration, and time, but can be appropriately selected by those skilled in the art.

As used herein, the term “prevention” means any act of suppressing or delaying the onset of rheumatoid arthritis by administration of the pharmaceutical composition according to the present disclosure.

As used herein, the term “treatment” means any act in which the symptoms of rheumatoid arthritis are alleviated or beneficially changed by administration of the pharmaceutical composition according to the present disclosure.

The term “alleviation,” as used herein, means any act of reducing parameters related to rheumatoid arthritis, for example, the severity of symptoms, by administration of the composition according to the present disclosure.

The pharmaceutical composition of the present disclosure is administered in a pharmaceutically effective amount. As used herein, the term “pharmaceutically effective amount” means an amount sufficient to treat the disease with a reasonable benefit/risk ratio applicable to medical treatment, and the effective dosage level can be determined according to factors such as the type and severity of the patient's disease, activity of the drug, sensitivity to the drug, timing of administration, route of administration and excretion rate, duration of treatment, concomitant drugs used, and other factors well known in the medical field.

The pharmaceutical composition of the present disclosure may be administered as a single therapeutic agent or in combination with other therapeutic agents, and may be administered sequentially or simultaneously with conventional therapeutic agents, either in single or multiple doses. It is important to administer an amount that provides maximum effect with minimal quantity and without side effects by taking into consideration all of the above factors, and such an amount can be easily determined by those skilled in the art.

Specifically, the effective amount of the pharmaceutical composition of the present disclosure may vary depending on the age, sex, condition, weight, absorption, inactivation rate, and excretion rate of the active ingredient in the body, type of disease, and concomitant drugs. In general, it may be administered daily or every other day at 0.001 to 150 mg per kg of body weight, preferably 0.01 to 100 mg, or divided into 1 to 3 doses per day. However, since the dosage may be increased or decreased depending on the route of administration, severity of rheumatoid arthritis, sex, weight, age, etc., the above dosage should not be construed as limiting the scope of the present disclosure in any way.

In another aspect, the present disclosure provides a method for preventing, controlling, or treating rheumatoid arthritis, the method comprising administering the pharmaceutical composition to a subject.

As used herein, the term “subject” refers to an object in need of a method for prevention, control, or treatment of a disease, and more specifically refers to mammals such as humans or non-human primates, mice, rats, dogs, cats, horses, and cattle.

The present disclosure can also provide a food composition including extracellular vesicles or 60 kDa chaperonin protein (Cpn60) derived from Propionibacterium freudenreichii strain as an active ingredient for the prevention or alleviation of rheumatoid arthritis.

More specifically, the composition of the present disclosure may be added to a health functional food for the purpose of preventing or alleviating rheumatoid arthritis. When the composition of the present disclosure is used as a food additive, it may be added as it is or used together with other foods or food ingredients, and may be appropriately used according to conventional methods. The amount of the active ingredient to be mixed may be suitably determined according to the intended purpose (prevention, health maintenance, or therapeutic treatment). Generally, when manufacturing food or beverages, the composition of the present disclosure is added in an amount of 15% by weight or less, preferably 10% by weight or less, based on the raw materials. However, for long-term intake for the purpose of health or hygiene maintenance or health regulation, the amount may be below the above range, and since there is no safety problem, the active ingredient may also be used in an amount exceeding the above range.

There is no particular limitation on the type of food. Examples of foods to which the above material may be added include meat, sausages, bread, chocolate, candies, snacks, confectionery, pizza, ramen, other noodles, chewing gum, dairy products including ice cream, various soups, beverages, teas, drinks, alcoholic beverages, and vitamin complexes, and include all health functional foods in the conventional sense.

The health drink composition of the present disclosure may further include, as additional components, various flavoring agents or natural carbohydrates like those of ordinary beverages. The natural carbohydrates may include monosaccharides such as glucose and fructose, disaccharides such as maltose and sucrose, polysaccharides such as dextrin and cyclodextrin, and sugar alcohols such as xylitol, sorbitol, and erythritol. As a sweetener, natural sweeteners such as thaumatin and stevia extract, or synthetic sweeteners such as saccharin and aspartame may be used. The ratio of the natural carbohydrate is generally about 0.01 to 0.20 g, preferably about 0.04 to 0.10 g, per 100 mL of the composition of the present disclosure.

In addition, the composition of the present disclosure may contain various nutrients, vitamins, electrolytes, flavoring agents, coloring agents, pectic acid and its salts, alginic acid and its salts, organic acids, protective colloid thickeners, pH regulators, stabilizers, preservatives, glycerin, alcohol, carbonating agents used in carbonated beverages, etc. Furthermore, the composition of the present disclosure may contain fruit pulp for the production of natural fruit juice, fruit juice beverages, and vegetable drinks. These components may be used independently or in combination. Although the proportion of these additives is not highly critical, they are generally selected in the range of 0.01 to 0.20 parts by weight per 100 parts by weight of the composition of the present disclosure.

As confirmed in an embodiment of the present disclosure, the extracellular vesicles or 60 kDa chaperonin protein (Cpn60) derived from Propionibacterium freudenreichii MJ2 strain have the effect of inhibiting the production of inflammatory cytokines, and thus can be used as a cosmetic composition having anti-inflammatory activity.

Accordingly, the present disclosure can provide an anti-inflammatory cosmetic composition including extracellular vesicles or 60 kDa chaperonin protein (Cpn60) derived from Propionibacterium freudenreichii MJ2 strain as an active ingredient.

In addition to extracellular vesicles or 60 kDa chaperonin protein (Cpn60) derived from Propionibacterium freudenreichii MJ2 strain as an active ingredient, the cosmetic composition of the present disclosure may further include components conventionally used in cosmetic compositions, for example, conventional adjuvants such as antioxidants, stabilizers, solubilizers, vitamins, pigments, and perfumes, as well as carriers.

The cosmetic composition of the present disclosure may be prepared in any formulation conventionally produced in the art, for example, solutions, suspensions, emulsions, pastes, gels, creams, lotions, powders, soaps, surfactant-containing cleansers, oils, powder foundations, emulsion foundations, wax foundations, and sprays, but is not limited thereto. More specifically, the cosmetic composition of the present disclosure may be formulated as a softening toner, nourishing toner, nourishing cream, massage cream, essence, eye cream, cleansing cream, cleansing foam, cleansing water, pack, spray, or powder.

When the formulation of the present disclosure is a paste, cream, or gel, carriers such as animal oils, vegetable oils, waxes, paraffins, starch, tragacanth, cellulose derivatives, polyethylene glycol, silicone, bentonite, silica, talc, or zinc oxide may be used. When the formulation is a solution or emulsion, carriers such as solvents, solubilizers, or emulsifiers may be used, for example, water, ethanol, isopropanol, ethyl carbonate, ethyl acetate, benzyl alcohol, benzyl benzoate, propylene glycol, 1,3-butylene glycol oil, glycerol aliphatic esters, polyethylene glycol, or fatty acid esters of sorbitan. When the formulation is a suspension, carriers such as liquid diluents including water, ethanol, or propylene glycol, suspending agents such as ethoxylated isostearyl alcohol, polyoxyethylene sorbitol esters, and polyoxyethylene sorbitan esters, microcrystalline cellulose, aluminum metahydroxide, bentonite, agar, or tragacanth may be used. When the formulation is a powder or spray, carriers such as lactose, talc, silica, aluminum hydroxide, calcium silicate, or polyamide powder may be used, and in the case of a spray, a propellant such as chlorofluorohydrocarbon, propane/butane, or dimethyl ether may additionally be included. When the formulation is a surfactant-containing cleansing agent, carriers such as aliphatic alcohol sulfates, aliphatic alcohol ether sulfates, sulfosuccinate monoesters, isethionates, imidazolinium derivatives, methyl taurates, sarcosinates, fatty acid amide ether sulfates, alkylamido betaines, aliphatic alcohols, fatty acid glycerides, fatty acid diethanolamides, vegetable oils, lanolin derivatives, or ethoxylated glycerol fatty acid esters may be used.

### MODE FOR CARRYING OUT THE INVENTION

The disclosure may be used for providing A better understanding of the present disclosure may be obtained through the following examples that are set forth to illustrate, but are not to be construed to limit, the scope of the present disclosure.

### Example 1

Isolation of Extracellular Vesicles Derived from Propionibacterium freudenreichii MJ2 Strain

The Propionibacterium freudenreichii MJ2 strain used in the present disclosure was the strain deposited by the inventors at the Korean Culture Center of Microorganisms on Jun. 8, 2018, and assigned accession number KCCM12272P. P. freudenreichii MJ2 was identified by selecting candidate strains through a single-colony isolation from raw milk derived from a farm in Gimpo, process Gyeonggi-do using a selective medium (YELA), followed by 16s rDNA sequencing.

In order to isolate extracellular vesicles from Propionibacterium freudenreichii MJ2 strain, MJ2, which had been activated at least twice on YELA medium, was inoculated and cultured at 30° C. for 48 hours under anaerobic conditions created using a GasPak (BD, Franklin Lakes, NJ, USA). The cultured broth was centrifuged at 10,000×g for 20 minutes at 4° C., and only the supernatant was filtered using a 0.22 μm vacuum filter. Thereafter, ultrafiltration (Amicon® Stirred Ultrafiltration Cell, Millipore, USA) using a filter with a molecular weight cutoff (MWCO) of 100 kDa was performed to separate extracellular vesicles larger than 100 kDa. The separated and concentrated solution was then filtered again using a 0.22 μm vacuum filter to remove debris and cell remnants. The obtained solution was subjected to ultracentrifugation (CP100WX, Hitachi, Tokyo, Japan) at 150,000×g for 3 hours at 4° C. to isolate extracellular vesicles. The pellet was resuspended in phosphate-buffered saline (PBS) and ultracentrifuged again at 150,000×g for 3 hours at 4° C. to remove medium components, thereby obtaining extracellular vesicles from Propionibacterium freudenreichii MJ2 strain.

### Example 2

Confirmation of Extracellular Vesicles Derived from Propionibacterium freudenreichii MJ2 Strain

In order to confirm the extracellular vesicles isolated from Propionibacterium freudenreichii MJ2 strain in Example 1, the size and morphology were analyzed using nanoparticle tracking analysis (NTA), scanning electron microscopy (SEM), and transmission electron microscopy (TEM).

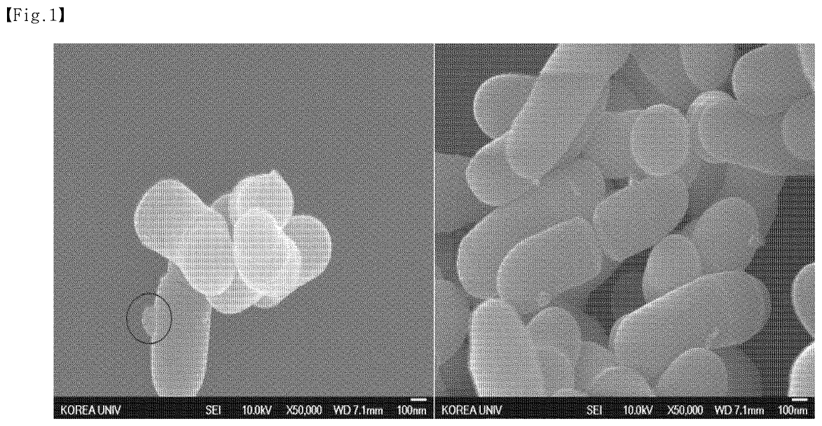

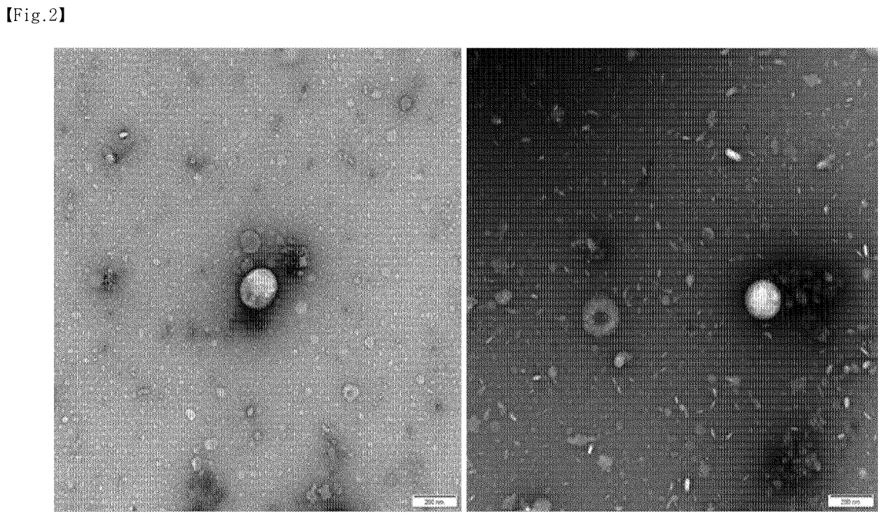

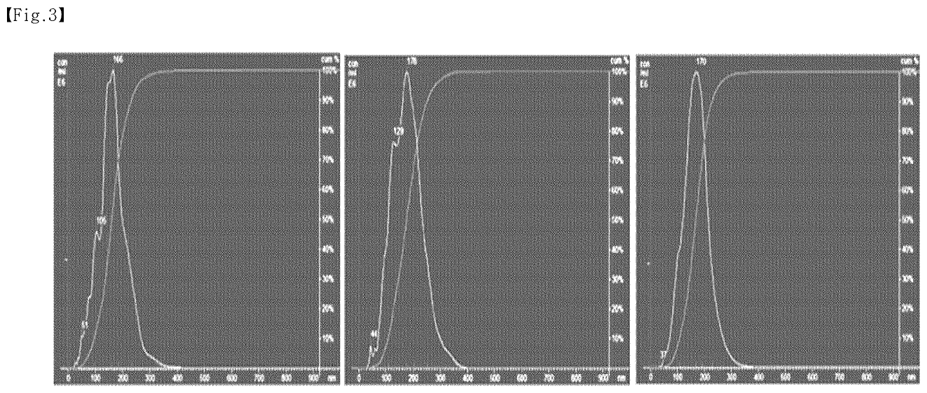

As a result, as shown in FIGS. 1 to 3, it was confirmed that the extracellular vesicles isolated in the present disclosure had a size of about 170 nm and were spherical in shape.

### Example 3

Protein Identification of Extracellular Vesicles Derived from Propionibacterium freudenreichii MJ2 Strain

For proteomic analysis of the extracellular vesicles derived from Propionibacterium freudenreichii MJ2 strain isolated in the present disclosure, the inventors performed in-solution trypsin digestion. Specifically, 100 μg of the extracellular vesicles isolated from Propionibacterium freudenreichii MJ2 strain in Example 1 was dissolved in a lysis solution containing 8 M urea (Sigma, St. Louis, MO, USA) and 1% protease inhibitor (Thermo Scientific, Waltham, MA, USA). Denaturation and reduction were carried out by adding 8 M urea and 25 mM DTT (Sigma, St. Louis, MO, USA) and reacting at 60° C. for 45 minutes. The sample was then cooled, and 25 mM iodoacetamide solution dissolved in 25 mM ammonium bicarbonate was added and reacted for 1 hour at room temperature in the dark. The urea was diluted to a final concentration of 1 M using 25 mM ammonium bicarbonate, and the eightfold-diluted proteins were digested overnight at 37° C. using Pierce trypsin protease (MS grade, Thermo Scientific, Waltham, MA, USA). After completion of the reaction, salts were removed from the sample using a C18 column (Oasis® HLB 1 cc Vac cartridges, Waters Corporation, MA, USA), and nano-liquid chromatography tandem mass spectrometry (Nano-LC-MS/MS) was performed using an Easy n-LC (Thermo Fisher, San Jose, CA, USA) coupled to an LTQ Orbitrap XL mass spectrometer (Thermo Fisher, San Jose, CA, USA). The digested peptides of the extracellular vesicles obtained by the in-solution digestion method were loaded onto a C18 nanobore column (150 mm×0.1 mm, 3 μm particle size) and separated under the following conditions: 0-40% buffer B for 80 minutes, 40-60% buffer B for 4 minutes, 95% buffer B for 4 minutes, 100% buffer A for 6 minutes; flow rate of 500 nL/min (buffer A composition: 0.1% formic acid, 3% acetonitrile in H₂O; buffer B composition: 0.1% formic acid in acetonitrile).

Mass spectrometry spectra were acquired using data-dependent acquisition consisting of a full mass scan (350-1200 m/z) followed by 10 MS/MS scans. Protein analysis of the extracellular vesicles derived from MJ2 strain was performed using the Mascot algorithm (Matrixscience, USA) to identify peptide sequences present in the protein sequence database.

As a result of proteomic analysis of the extracellular vesicles using the above method, 20 proteins as listed in Table 1 were identified, and it was found that 60 kDa chaperonin, Chain A, and methylmalonyl-CoA carboxyltransferase 12S subunit protein were predominant.

TABLE 1
Proteomic Analysis for Extracellular Vesicles

| | | Protein | Monoisotopic | |
|---|---|---|---|---|
| Accession | Protein description | score | mass (Mr) | pI |
| CEH03981.1 | 60 kDa chaperonin 1 | 362 | 56244 | 4.70 |
| | (Protein Cpn60 1) | | | |
| | (groEL protein 1) | | | |
| | (Heat shock protein | | | |
| | 60 1) | | | |
| pdb\|1ON3\|A | Chain A, | 334 | 334 | 5.38 |
| | Methylmalonyl-CoA | | | |
| | carboxyltransferase | | | |
| | 12S subunit | | | |
| AAA25676.1 | methylmalonyl-CoA | 289 | 66057 | 5.22 |
| | carboxyltransferase | | | |
| | subunit 12S monomer | | | |
| pdb\|1RR2\|A | Chain A, | 164 | 52623 | 5.64 |
| | transcarboxylase | | | |
| | 5S subunit | | | |
| CEH00767.1 | penicillin-binding | 130 | 75382 | 7.71 |
| | protein | | | |
| | (peptidoglycan | | | |
| | glycosyltransferase) | | | |
| CEH00696.1 | iron-sulfur protein | 107 | 57776 | 6.75 |
| CEH01375.1 | dolichyl-phosphate | 86 | 32123 | 5.34 |
| | beta-D- | | | |
| | mannosyltransferase | | | |
| CEH02479.1 | surface layer | 78 | 58552 | 4.69 |
| | protein A (S-layer | | | |
| | protein A) | | | |
| CEH00694.1 | Hypothetical protein | 77 | 23227 | 4.70 |
| | PFCIRM127_12145 | | | |
| CEH00758.1 | Carboxylic ester | 74 | 46724 | 5.38 |
| | hydrolase | | | |
| CEH00733.1 | Methylmalonyl-CoA | 73 | 12360 | 5.39 |
| | carboxytransferase, | | | |
| | 1.3S subunit | | | |
| | (Transcarboxylase, | | | |
| | 1.3S subunit). 123 bp | | | |
| CEH02032.1 | Enolase 1 | 66 | 46005 | 4.47 |
| CEH02295.1 | 50S ribosomal | 57 | 30408 | 11.08 |
| | protein L2 | | | |
| CEH04045.1 | Hypothetical protein | 44 | 10739 | 6.59 |
| | PFCIRM125_11275 | | | |
| CEH00999.1 | dihydrolipoyllysine- | 42 | 48357 | 5.58 |
| | residue (2- | | | |
| | methylpropanoyl) | | | |
| | transferase. Lipoamide | | | |
| | acyltransferase | | | |
| | component of | | | |
| | branched-chain | | | |
| | alpha-keto acid | | | |
| | dehydrogenase | | | |
| | complex (E2) | | | |
| | (Dihydrolipoamide | | | |
| | branched chain | | | |
| | transacylase) | | | |
| CEH00698.1 | Oxidoreductase | 39 | 29476 | 5.56 |
| CEH00239.1 | Putative minor | 39 | 150361 | 5.25 |
| | extracellular serine | | | |
| | protease | | | |
| CEH01003.1 | 2-oxoisovalerate | 38 | 40647 | 4.60 |
| | dehydrogenase | | | |
| | subunit alpha | | | |
| | (Branched-chain | | | |
| | alpha-keto acid | | | |
| | dehydrogenase E1 | | | |
| | component alpha | | | |
| | chain) (BCKDH | | | |
| | E1-alpha) | | | |
| CEH02678.1 | Hypothetical protein | 35 | 35910 | 5.29 |
| | PFCIRM125_06735 | | | |
| CEH01770.1 | polar amino acid ABC | 33 | 31672 | 4.84 |
| | transporter, binding | | | |
| | protein component | | | |

### Example 4

Cytotoxicity Analysis of Extracellular Vesicles Derived from Propionibacterium freudenreichii MJ2 Strain

To confirm whether the extracellular vesicles derived from Propionibacterium freudenreichii MJ2 strain according to the present disclosure cause cytotoxicity, RAW 264.7 cells were seeded in 96-well plates at a density of 1×10⁴ cells/mL and cultured for 24 hours in a 5% CO₂ incubator at 37° C. After 24 hours, the extracellular vesicles of the present disclosure, diluted in serum-free DMEM (Dulbecco's Modified Eagle Medium), were treated at concentrations of 1×10⁶, 1×10⁷, 1×10⁸, and 1× 10⁹ particles/mL, and cultured for 4 days. Cell viability was then measured using MTT reagent with a SpectraMax 340PC384 microplate reader (Molecular Devices, Sunnyvale, CA, USA).

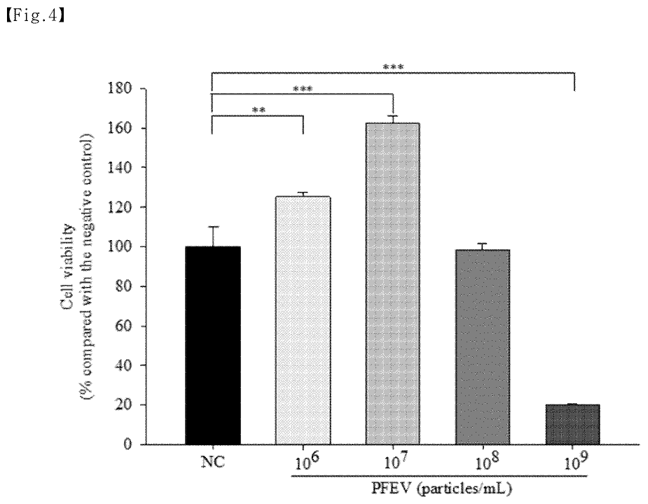

As shown in FIG. 4, compared with the control group cultured in DMEM medium without treatment of the extracellular vesicles of the present disclosure, the experimental groups treated with 1×10⁶ to 1×10⁸ particles/mL of extracellular vesicles showed more than 90% cell viability, confirming that the extracellular vesicles isolated from the MJ2 strain of the present disclosure do not cause cytotoxicity in cells.

### Example 5

Analysis of the Inhibitory Activity of Extracellular Vesicles Derived from Propionibacterium freudenreichii MJ2 Strain on Osteoclast Differentiation

In order to confirm whether the extracellular vesicles derived from Propionibacterium freudenreichii MJ2 strain isolated in Example 1 have osteoclast the differentiation inhibitory activity, following experiments were conducted.

### <5-1> Inhibition of Number of TRAP (Tartrate-Resistant Acid Phosphatase)-Activated Osteoclasts

RAW 264.7 macrophage cell line was cultured in DMEM (Dulbecco's Modified Eagle Medium) medium supplemented with 10% FBS, 100 units/mL penicillin, and 100 μg/mL streptomycin under 5% CO₂-95% air composition at 37° C. When the cells reached 80% confluence, they were detached from the culture plate using a scraper, adjusted to a density of 1×10⁴ cells/mL, stabilized for one day in an incubator at 37° C., and then the medium was replaced with α-MEM (Minimum Essential Medium Eagle-Alpha Modification) supplemented with 10% FBS, 100 units/mL penicillin, and 100 μg/mL streptomycin. To induce differentiation of the macrophages into osteoclasts, RANKL (Receptor activator of nuclear factor kappa-B ligand) was at added a concentration of 50 ng/mL. Subsequently, the extracellular vesicles according to the present disclosure were additionally treated at concentrations of 1×10⁶, 1×10⁷, and 1×10⁸ particles/mL and cultured for 4 days.

Thereafter, to confirm the activity of TRAP, which is an osteoclast marker, the number of differentiated osteoclasts was measured by TRAP staining. The supernatant was removed from each group of cells, the cells were washed with PBS, and then fixed. The fixed cells were stained for 30 minutes at 37° C. using a TRACP & ALP Assay Kit (Takara, Shiga, Japan). After removing the supernatant and washing the cells with distilled water, osteoclasts with three or more nuclei, i.e., differentiated osteoclasts, were observed under an optical microscope.

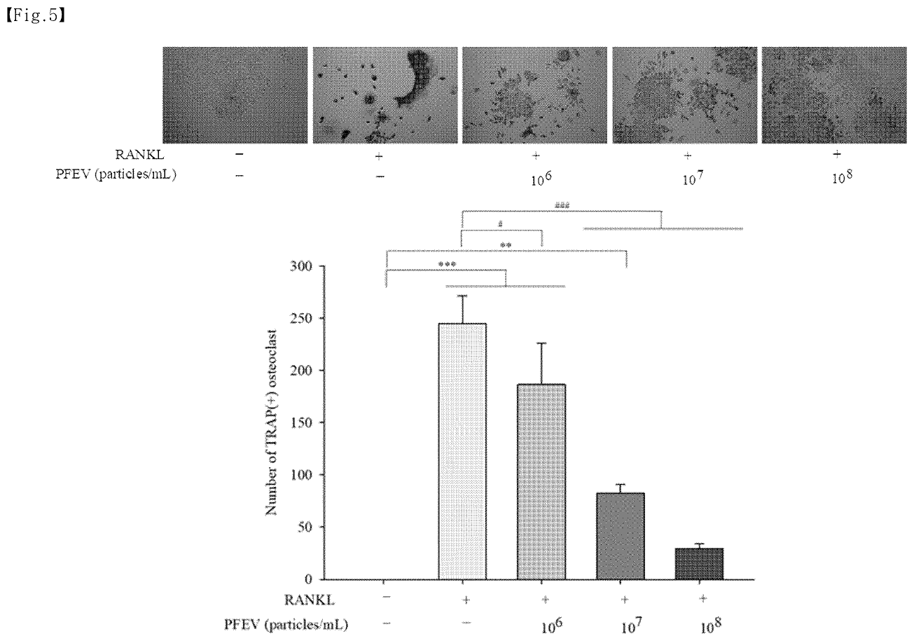

As shown in FIG. 5, in the groups treated with the extracellular vesicles of the present disclosure, the number of TRAP-activated osteoclasts was significantly decreased compared with the control group treated with RANKL alone without extracellular vesicles.

These results confirmed that the extracellular vesicles derived from Propionibacterium freudenreichii MJ2 strain have activity to inhibit osteoclast differentiation.

### <5-2> Inhibition of Gene Expression of Osteoclast Differentiation-Inducing Factors

From the cells cultured in the same manner as in Example 5-1, the supernatant was removed, and RNA was extracted from each cell group using Ribo-Ex (GeneAll Biotechnology, Seoul, Korea). The isolated RNA was synthesized into cDNA using the RevertAid First Strand CDNA kit (Thermo Scientific, Waltham, MA, USA), and real-time PCR was performed using the KAPA SYBR FAST qPCR kit (Kapa Biosystems, Foster City, CA, USA) on a QuantStudio 6 Flex (Life Technologies, Thermo Scientific) instrument. The primer information used is shown in Table 2 below.

TABLE 2
Information on Primers for Genes Used in Real-Time
PCR

| Gene | Forward (5′-3′) | Reverse (5′-3′) |
|---|---|---|
| GAPDH | ACCCAGAAGACTGTGGATGG | CACATTGGGGGTAGGAACAC |
| RANK | TGCAGCTCAACAAGGATACG | GAGCTGCAGACCACATCTGA |
| NFKB | TCCTGGCCTCTAGCCTTGTA | GCCAAGGAAGAAAAGTGCTG |
| c-fos | CCAGTCAAGAGCATCAGCAA | AAGTAGTGCAGCCCGGAGTA |
| NFATc1 | GGTGCTGTCTGGCCATAACT | GCGGAAAGGTGGTATCTCAA |
| Calcr | CGGACTTTGACACAGCAGAA | GTCACCCTCTGGCAGCTAAG |
| Ctsk | CAGCTTCCCCAAGATGTGAT | AGCACCAACGAGAGGAGAAA |
| Atp6v0d2 | GACCCTGTGGCACTTTTTGT | GCTTGCATTTGGGGAATCTA |

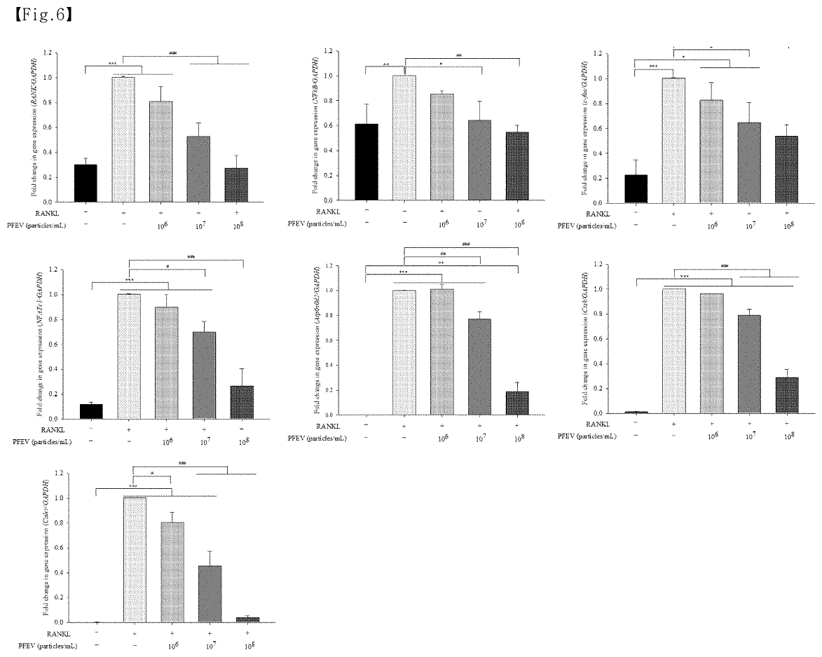

As shown in FIG. 6, the groups treated with 1×10⁷ particles/mL and 1×10⁸ particles/mL of the extracellular vesicles of the present disclosure expressed genes involved in promoting osteoclast differentiation at significantly reduced levels compared with the control group treated only with RANKL.

### <5-3> Inhibition of Protein Expression of Osteoclast Differentiation-inducing Factors

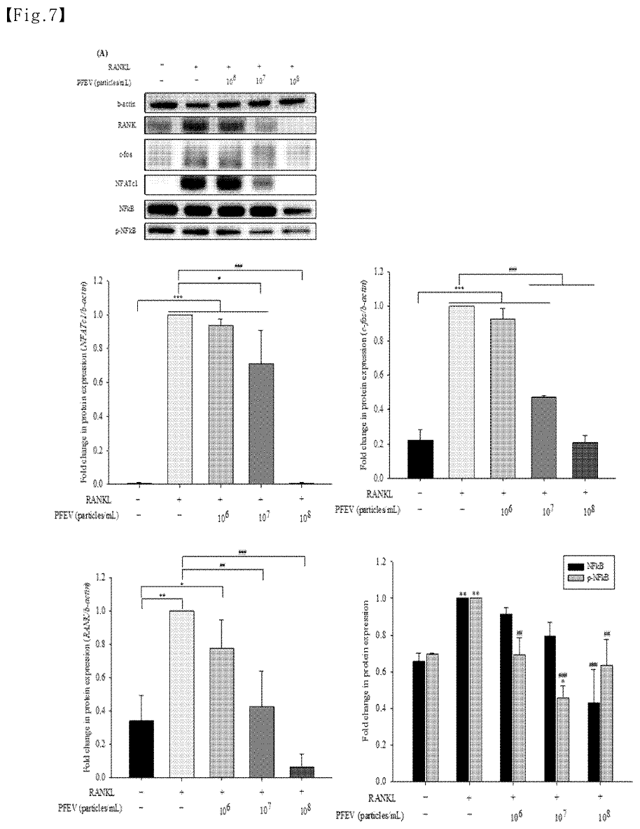

The cells used in Example 5-2 were centrifuged at 2,500×g for 5 minutes at 4° C. to remove the supernatant and collect the cells. The cells were washed with cold PBS and lysed on ice for 15 minutes using RIPA lysis buffer (BYLABS, Korea) supplemented with 1% protease inhibitor cocktail (Thermo Scientific). The lysed cells were centrifuged at 14,000×g for 15 minutes at 4° C., and the resulting supernatant was used as the protein sample. The protein content was quantified using the Pierce BCA Protein Assay kit (Thermo Scientific), denatured at 100° C., and subjected to Western blotting. Ten micrograms of protein were loaded onto a 10% SDS-polyacrylamide gel, separated by SDS-PAGE, transferred to a PVDF membrane, reacted with specific antibodies, and protein bands were detected using a UVP ChemStudio (Analytik, Jena, Germany). As shown in FIG. 7, the expression levels of c-fos, RANK, and NFATc1 were significantly increased in the control group induced with RANKL to promote osteoclast differentiation compared with the group without RANKL induction. In contrast, in the experimental groups treated with the extracellular vesicles of the present disclosure, the expression levels of c-fos, RANK, and NFATc1 were significantly decreased in a concentration-dependent manner compared with the untreated group.

These results indicate that the extracellular vesicles derived from Propionibacterium freudenreichii MJ2 strain according to the present disclosure can not only inhibit the expression of genes that induce osteoclast differentiation, which is a major cause of rheumatoid arthritis, but also inhibit the expression of proteins involved in osteoclast differentiation, thereby preventing, improving, and treating the onset of rheumatoid arthritis caused by osteoclast differentiation.

### Example 6

Alleviative Effect on Rheumatoid Arthritis by Administration of Extracellular Vesicles Derived from Propionibacterium freudenreichii MJ2 Strain

### <6-1> Preparation of Collagen-Induced Arthritis (CIA) Mouse Model

Male DBA/1J mice (8 weeks old, Orient Bio, Seoul, Korea) were used to prepare a collagen-induced arthritis (CIA) mouse model. After a 1-week acclimatization period, the mice were randomly assigned into groups of eight animals each: a group injected with PBS without arthritis induction (Control), a group injected with PBS after arthritis induction (Model), a group administered a low dose of extracellular vesicles derived from P. freudenreichii MJ2 (1×10⁷ particles/mouse) after arthritis induction (Low-dose Extracellular Vesicles; LDEV), and a group administered a high dose of extracellular vesicles derived from P. freudenreichii MJ2 (1×10⁸ particles/mouse) after arthritis induction (High-dose Extracellular Vesicles; HDEV). Arthritis was induced by emulsifying type II bovine collagen (2 mg/mL) with complete Freund's adjuvant (CFA, 1 mg/mL) at a 1:1 ratio and intradermally injecting it into the tail to perform the first immunization (1st immunization). Three weeks later, type II bovine collagen (2 mg/mL) was emulsified with incomplete Freund's adjuvant (IFA) at a 1:1 ratio and intradermally injected into the tail to perform the second immunization (2nd boost). After arthritis induction, the severity of swelling and inflammation was visually evaluated according to the arthritic score criteria shown in Table 3 below.

TABLE 3
Arthritic score criteria

| | |
|---|---|
| Score 0 | No signs of arthritis such as swelling, |
| | erythema, or edema |
| Score 1 | Mild swelling and erythema or one to two |
| | inflamed joints |
| Score 2 | Definite erythema, swelling, or edema in |
| | more than two joints |
| Score 3 | Severe swelling and edema from toes to |
| | ankle, with impaired free movement |

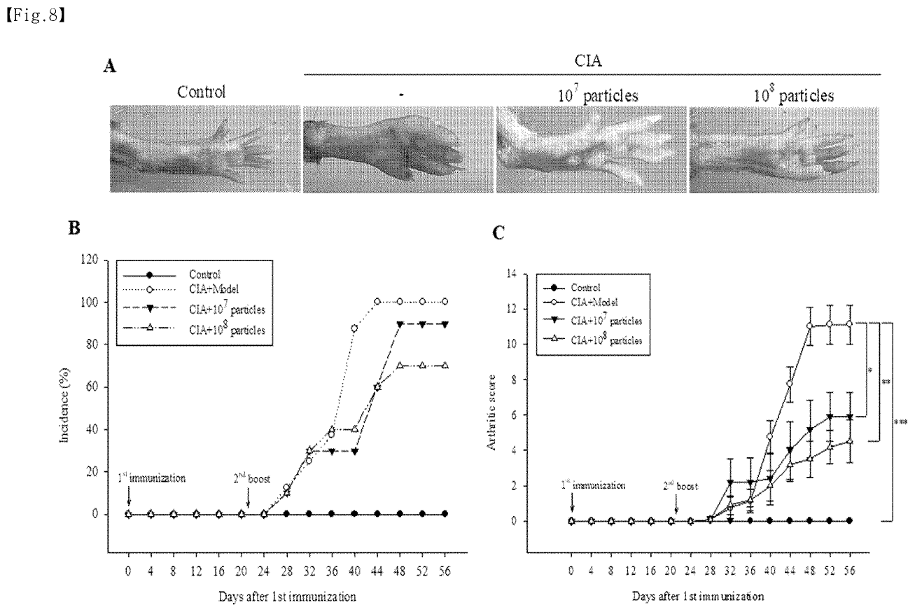

As shown in FIG. 8, the group administered PBS after arthritis induction exhibited the most severe arthritis symptoms, whereas the groups administered extracellular vesicles derived from P. freudenreichii MJ2 strain showed a visually significant improvement in arthritis symptoms.

### <6-2> Analysis of Hepatotoxicity Indicators in CIA Mice

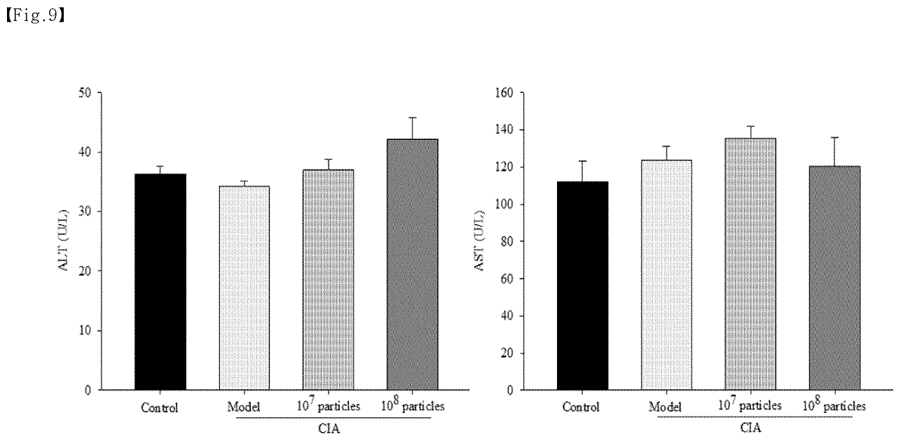

Serum levels of AST (aspartate aminotransferase) and ALT (alanine aminotransferase), which are indicators of hepatotoxicity, were measured. As shown in FIG. 9, no significant difference was observed between the experimental groups. These results confirmed that administration of the extracellular vesicles within the dose range used in the present disclosure does not cause hepatotoxicity in mice.

### <6-3> Analysis of Effect on Bone Erosion in Rheumatoid Arthritis Mouse Model by Administration of Extracellular Vesicles of the Present Disclosure

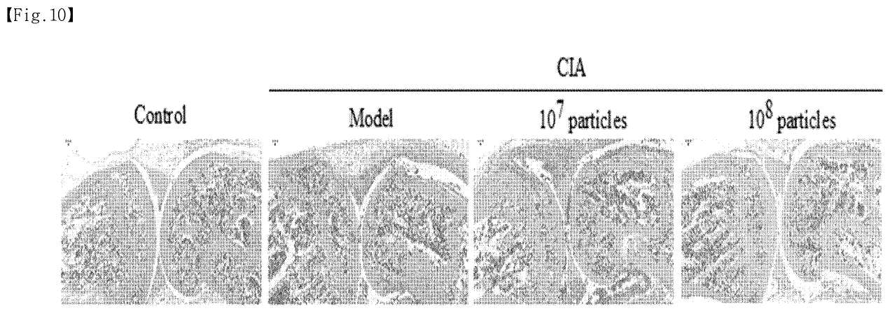

H&E staining was performed to compare bone erosion and knee joint damage. As a result, it was confirmed that administration of the extracellular vesicles of the present disclosure reduced knee joint damage, and that bone erosion and knee joint damage could be alleviated when the extracellular vesicles were administered to collagen-induced arthritis mice (see FIG. 10).

<6-4> Analysis of Collagen Antigen-Specific IgG, IgG1, and IgG2a Antibody Concentrations in Collagen-Induced Rheumatoid Arthritis Mice Administered with the Extracellular Vesicles of the Present Disclosure

Blood was collected from the mice used in Example 6-1, plasma was isolated, and the concentrations of collagen antigen-specific IgG, IgG1, and IgG2a antibodies were measured by ELISA.

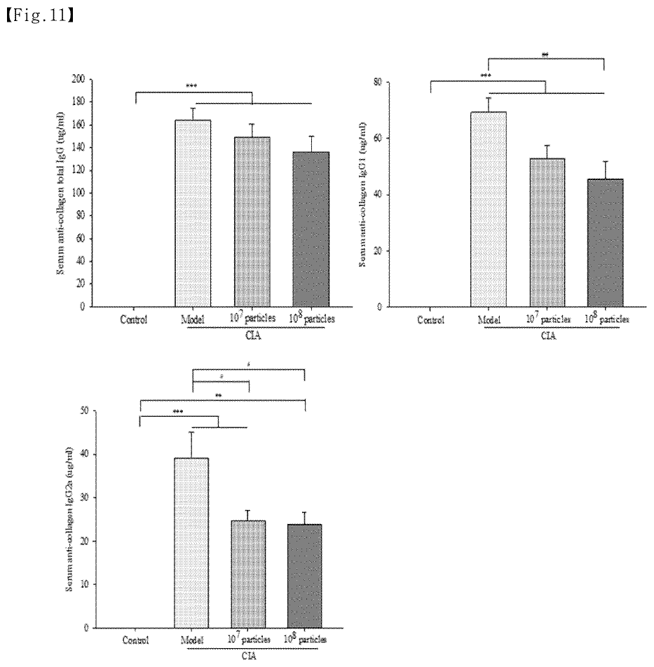

As shown in FIG. 11, production of collagen antigen-specific IgG antibodies was increased in the blood upon induction of arthritis, whereas in the groups administered the extracellular vesicles of the present disclosure, the concentrations of total IgG, IgG1, and IgG2a antibodies specific to collagen antigen were significantly decreased.

<6-5> Analysis of Immune Response and Changes in Inflammatory Cytokines in Collagen-Induced Rheumatoid Arthritis Mice Administered with the Extracellular Vesicles of the Present Disclosure

Blood was collected from the mice used in Example 6-1, and serum was used to analyze levels of inflammatory cytokines TNFα and IL-6 by ELISA, and mRNA expression levels of IL-17 were analyzed by quantitative real-time PCR. The amount of anti-inflammatory cytokine IL-10 was also measured by ELISA.

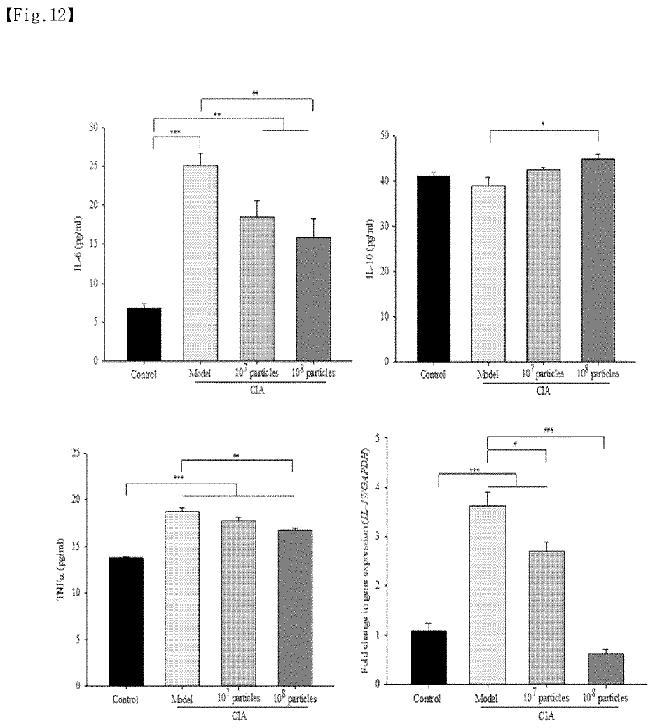

As shown in FIG. 12, levels of inflammatory cytokines TNFα, IL-6, and IL-17 were significantly reduced in the group administered high-dose extracellular vesicles of the present disclosure compared with the arthritis-induced group. In the case of IL-10, the group administered high-dose extracellular vesicles of the present disclosure showed a tendency toward increased expression.

Accordingly, these results confirmed that administration of the extracellular vesicles of the present disclosure can suppress excessive immune responses caused by rheumatoid arthritis, suppress levels of inflammatory cytokines, and increase levels of anti-inflammatory cytokines.

### <6-6> Analysis of the Effect of Extracellular Vesicle Administration on Osteoclast Differentiation in Collagen-Induced Rheumatoid Arthritis Mice

Synovium tissue was isolated from the mice used in Example 6-1, and the relative mRNA expression levels of factors related to osteoclast differentiation, including nuclear factor of activated T cells 1 (NFATc1), cathepsin K (Ctsk), calcitonin receptor (Calcr), matrix metallopeptidase 9 (MMP9), osteoprotegerin (OPG), and receptor activator of nuclear factor-KB ligand (RANKL), were analyzed by quantitative real-time PCR.

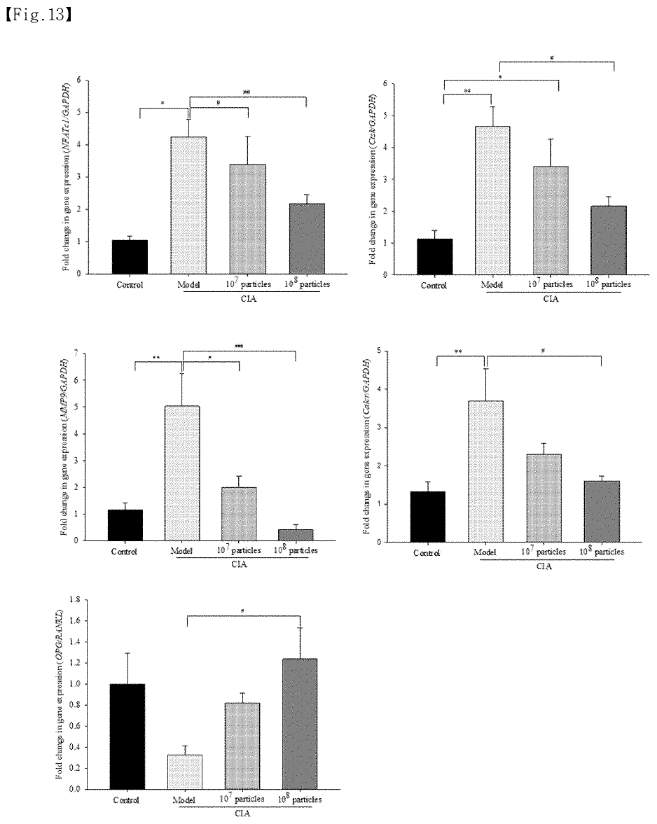

As shown in FIG. 13, the expression levels of NFATc1, Ctsk, Calcr, and MMP9 were significantly increased in the arthritis-induced group compared with the non-induced group, whereas in the groups administered the extracellular vesicles of the present disclosure, NFATc1 and MMP9 expression levels were significantly decreased at all doses, and Ctsk and Calcr expression levels were significantly decreased at high doses. Furthermore, the OPG/RANKL expression ratio, which indicates inhibition of osteoclast differentiation and promotion of osteoblast differentiation, was significantly increased in the group administered high-dose extracellular vesicles derived from P. freudenreichii MJ2 strain.

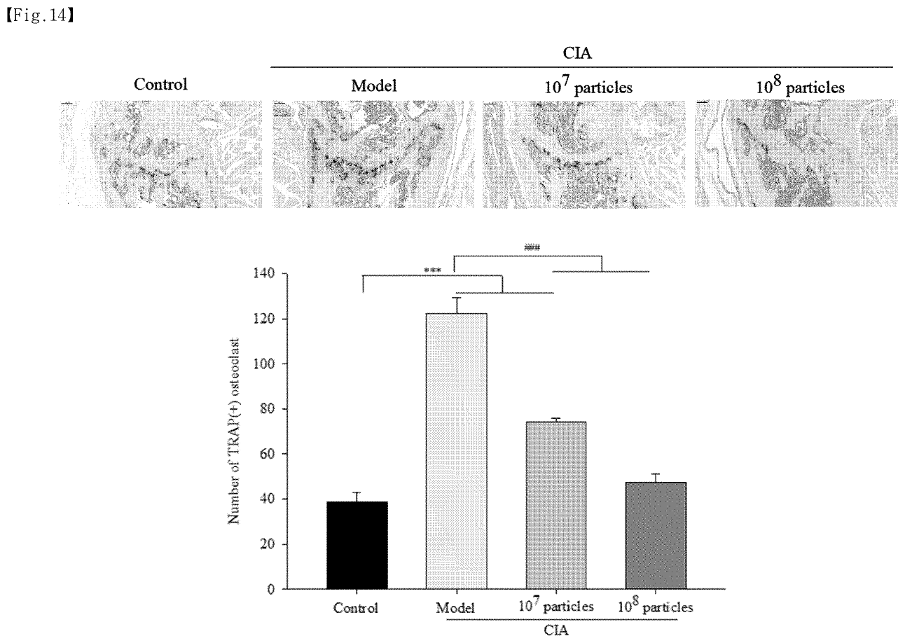

In addition, TRAP staining of the knee joint tissue showed that the number of TRAP-positive cells (osteoclasts) stained red-purple was markedly increased in the arthritis-induced group compared with the non-induced group. However, in the groups administered the extracellular vesicles of the present disclosure, the number of TRAP-positive cells was significantly decreased compared with the arthritis-induced group (see FIG. 14).

These results demonstrated that the extracellular vesicles of the present disclosure isolated from Propionibacterium freudenreichii MJ2 strain can effectively inhibit osteoclast differentiation and improve inflammatory responses, thereby preventing, improving, and treating rheumatoid arthritis.

### Example 7

Production of 60 kDa Chaperonin Protein (Cpn60) Contained in the Extracellular Vesicles Derived from Propionibacterium freudenreichii MJ2 Strain

As confirmed in Example 3, the inventors identified major proteins contained in the extracellular vesicles derived from Propionibacterium freudenreichii MJ2 strain by proteomic analysis, and confirmed that 60 kDa chaperonin protein (Cpn60) was a major component of the extracellular vesicles in Table 1. Accordingly, the inventors synthesized the 60 kDa chaperonin protein (Cpn60) using an ExiProgen™ EC Protein Synthesis Kit and ExiProgen™ system (Bioneer, Daejeon, Korea), which are well known in the art, and then isolated and purified the synthesized Cpn60 protein.

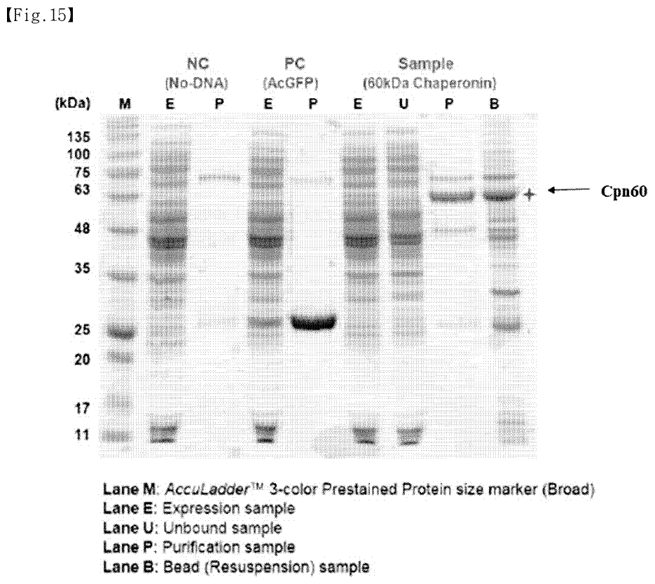

The purified Cpn60 protein was confirmed by SDS-PAGE and Coomassie blue staining, and as shown in FIG. 15, the purified Cpn60 protein was confirmed to be near 60 kDa.

### Example 8

Cytotoxicity Analysis of 60 kDa Chaperonin Protein (Cpn60) Derived from Propionibacterium freudenreichii MJ2 Strain

Cytotoxicity of the 60 kDa chaperonin protein (Cpn60) purified in Example 7 was analyzed. RAW 264.7 cells were treated with RANKL (50 ng/ml) to induce differentiation into osteoclasts, and Cpn60 protein was treated at concentrations of 1.25-5 μg/mL, followed by cytotoxicity analysis using the MTT assay.

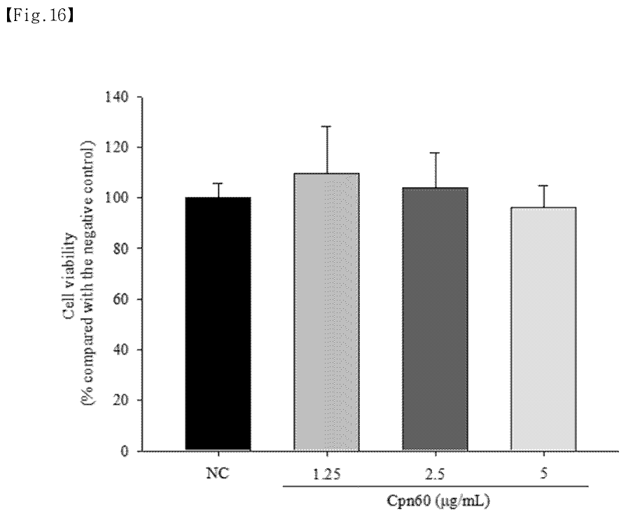

As shown in FIG. 16, no cytotoxicity was observed within the entire concentration range of 60 kDa chaperonin protein (Cpn60) treatment of the present disclosure.

### Example 9

Analysis of TRAP Enzyme Activity Inhibition by 60 kDa Chaperonin Protein (Cpn60) Derived from Propionibacterium freudenreichii MJ2 Strain

RAW 264.7 cells were treated with RANKL (50 ng/ml) to induce osteoclast differentiation, and Cpn60 protein was treated for 4 days. The osteoclast differentiation marker TRAP enzyme was then stained and observed.

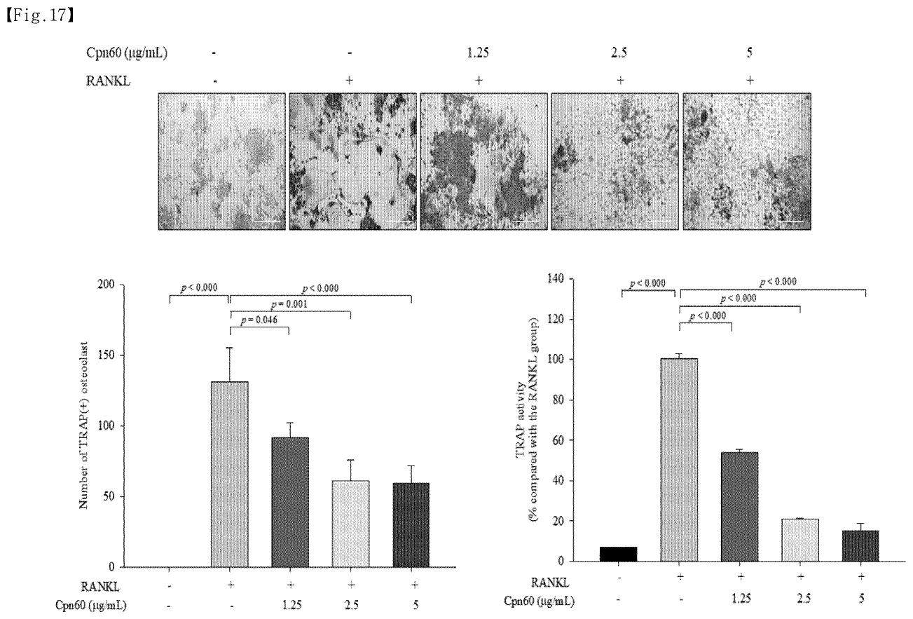

As shown in FIG. 17, the group treated with Cpn60 protein showed a significant decrease in the activity of TRAP enzyme, a marker of osteoclast differentiation.

### Example 10

Analysis of Inhibitory Effect of 60 kDa Chaperonin Protein (Cpn60) Derived from Propionibacterium Freudenreichii MJ2 Strain Against Expression of Genes Related to Osteoclast Differentiation

RAW 264.7 cells were treated with RANKL (50 ng/ml) to induce differentiation into osteoclasts, and Cpn60 was treated for 3 days. RNA was extracted from the cells of each experimental group, and the changes in expression levels of genes related to osteoclast differentiation were analyzed by real-time PCR.

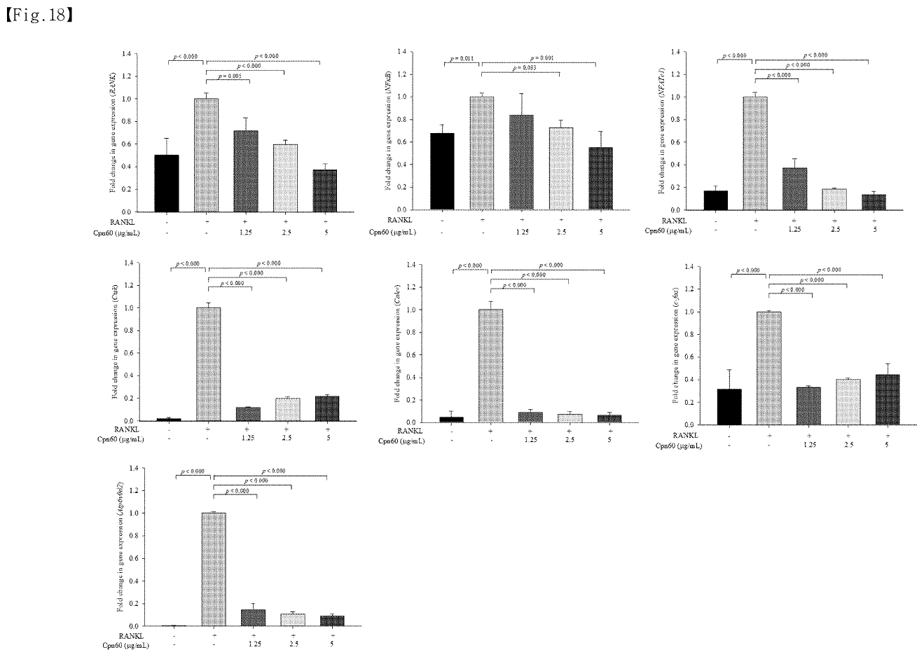

As shown in FIG. 18, in the group treated with Cpn60 protein, the expression levels of genes related to osteoclast differentiation-RANK, NFκB, c-fos, NFATc1, Calcr, Ctsk, and Atp6v0d2-were all significantly decreased compared with the group treated with RANKL alone.

### Example 11

Confirmation of Improvement Effect of Rheumatoid Arthritis by Administration of 60 kDa Chaperonin Protein Derived from Propionibacterium freudenreichii MJ2 Strain

### <11-1> Preparation of Collagen-Induced Arthritis (CIA) Mouse Model

Male DBA/1J mice (8 weeks old, Orient Bio, Seoul, Korea) were used as a collagen-induced arthritis (CIA) mouse model. After a 1-week acclimatization period, the mice were randomly divided into groups of 14 animals each: a group administered PBS without arthritis induction (Control), a group administered PBS after arthritis induction (Model), a group administered P. freudenreichii MJ2-derived surface protein fraction (1 mg/kg/mouse) prior to arthritis induction (SP), and a group administered Cpn60 (0.1 mg/kg/mouse) prior to arthritis induction (Cpn60). Each sample was administered for 3 weeks prior to arthritis induction by oral gavage (Control and Model groups) or intraperitoneal injection (SP and Cpn60 groups), and arthritis was induced with collagen. Arthritis induction was performed by emulsifying type II bovine collagen with complete Freund's adjuvant (CFA) at a 1:1 ratio, followed by intradermal injection into the tail for the first immunization (1st immunization), and three weeks later, type II bovine collagen was emulsified with incomplete Freund's adjuvant (IFA) at a 1:1 ratio and intradermally injected into the tail for the second immunization (2nd boost). After arthritis induction, the degree of swelling and inflammation was visually evaluated according to the arthritic score criteria (Table 3 above). For this arthritis animal model experiment, the surface protein fraction (SP) isolated from P. freudenreichii MJ2 strain, which showed osteoclast differentiation inhibitory effect in the in vitro evaluation, was used in combination to evaluate its effect.

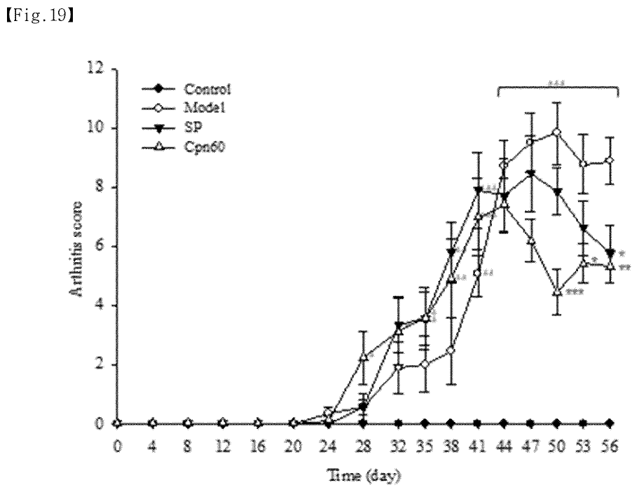

As shown in FIG. 19, the group administered PBS after arthritis induction exhibited the most severe arthritis symptoms. The group administered SP showed a statistically significant reduction in arthritis symptoms on day 56 after CIA induction, and the group administered Cpn60 showed a statistically significant reduction in arthritis symptoms on days 50, 53, and 56 after CIA induction.

### <11-2> Analysis of Body Weight and Hepatotoxicity Indicators in CIA Mice

For each animal group used in Example 11-1, body weight changes were measured weekly. Compared with the control group, the group in which arthritis was induced by collagen injection showed a significant decrease in body weight; however, there was no significant difference in body weight change among the arthritis-induced groups (Table 4).

TABLE 4
Body weight change

| | Initial | Final |
|---|---|---|
| Group | weight (mg) | weight (mg) |
| Control | 24.278 ± 0.798 | 27.389 ± 1.438 |
| Model | 23.511 ± 1.355 | 23.267 ± 1.669*** |
| SP | 23.667 ± 1.209 | 23.989 ± 1.801*** |
| Cpn60 | 23.100 ± 1.503 | 23.478 ± 1.138*** |

***p < 0.000, compared with the control group

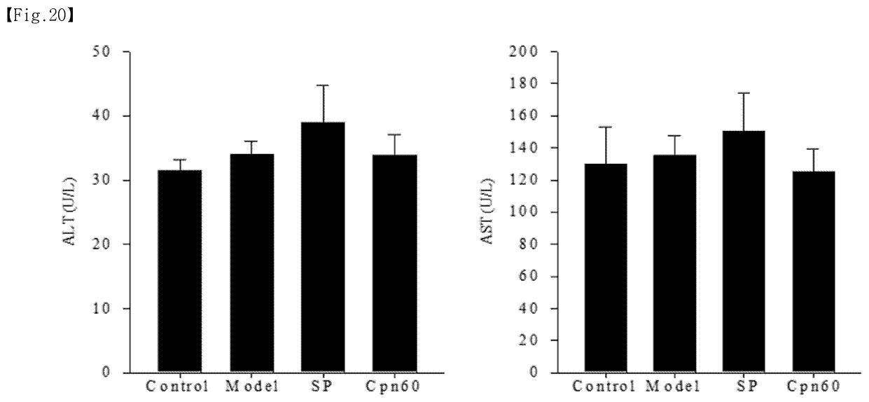

In addition, serum levels of AST (aspartate aminotransferase) and ALT (alanine aminotransferase), which are indicators of hepatotoxicity, were measured. As shown in FIG. 20, no significant difference was observed between the experimental groups. These results confirmed that within the dose ranges used in the present disclosure, administration of SP and Cpn60 does not cause hepatotoxicity in mice.

### <11-3> Analysis of the Effect of 60 kDa Chaperonin Protein (Cpn60) Administration on Bone Erosion in Rheumatoid Arthritis Mouse Model

To confirm bone erosion symptoms caused by rheumatoid arthritis, the hind paws of the mice used in Example 11-1 were analyzed using micro-CT.

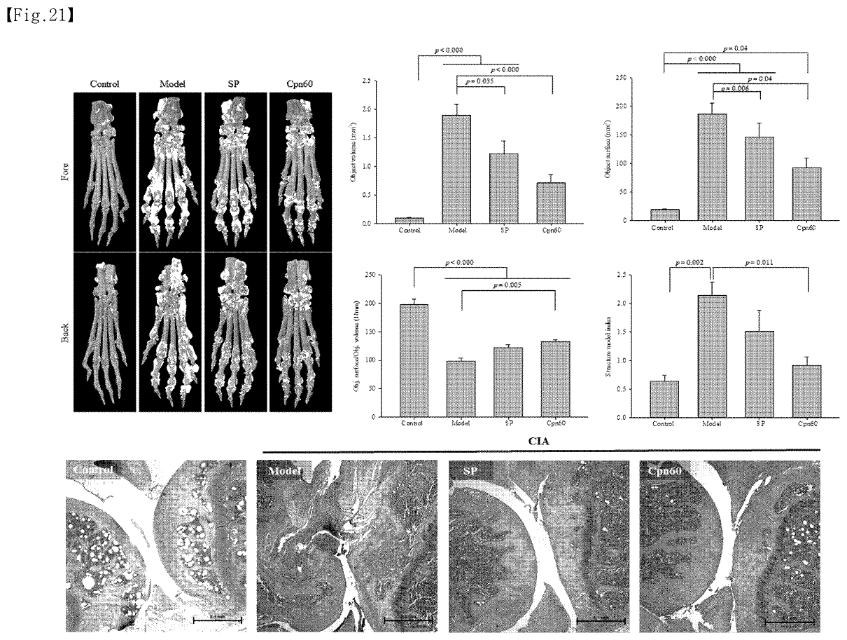

As shown in FIG. 21, compared with the control group in which rheumatoid arthritis was not induced, the collagen-induced arthritis group (model) exhibited evident bone erosion. Analysis using the micro-CT program revealed that both the volume and surface area of bone erosion were significantly decreased the group administered the Cpn60 protein compared with the arthritis-induced group.

In addition, the surface/volume ratio was significantly higher in the Cpn60-treated group compared with the Rheumatoid arthritis-induced group. In the structural model index used to evaluate trabecular bone rods and plates, Cpn60 treatment resulted in a statistically significant reduction compared with the arthritis-induced group.

Furthermore, H&E staining analysis comparing knee joint bone erosion and damage confirmed that knee joint damage was reduced by administration of the Cpn60 protein of the present disclosure, indicating that bone erosion and knee joint damage can be alleviated by administering Cpn60 protein to collagen-induced arthritis mice (see FIG. 21).

<11-4> Analysis of Collagen Antigen-specific IgG, IgG1, and IgG2a Antibody Concentrations in Collagen-induced Rheumatoid Arthritis Mice Administered with 60 kDa Chaperonin Protein (Cpn60)

Blood was collected from the mice used in Example 11-1, plasma was isolated, and collagen antigen-specific IgG, IgG1, and IgG2a antibody concentrations were measured by ELISA. Specifically, mouse type II collagen was coated on immunoplates at 4° C., blocked at room temperature, and then samples and horseradish peroxidase (HRP)-conjugated anti-mouse IgG, anti-IgG1, and anti-IgG2a antibodies were added and incubated for 3 hours. After 3 hours, substrate was added and the reaction was stopped with 2N H₂SO₄, and the amount of each IgG antibody in plasma was measured.

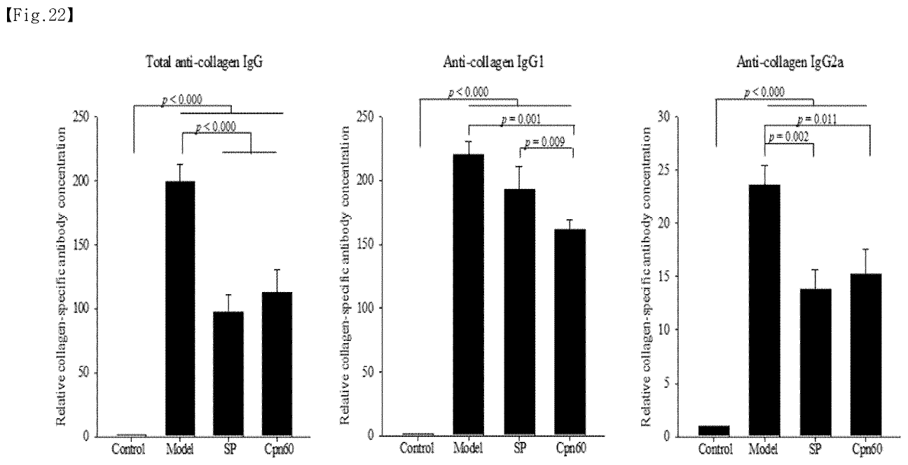

As shown in FIG. 22, production of collagen antigen-specific IgG antibodies in blood was increased upon arthritis induction, whereas in the groups administered the Cpn60 protein of the present disclosure, the concentrations of total IgG, IgG1, and IgG2a antibodies specific to collagen antigen were significantly decreased.

<11-5> Analysis of Immune Response and Changes in Inflammatory Cytokines in Collagen-induced Rheumatoid Arthritis Mice Administered with 60 kDa Chaperonin Protein (Cpn60)

Blood was collected from the mice used in Example 11-1, and serum was used to analyze levels of inflammatory cytokines TNFα and IL-6 by ELISA, and mRNA expression level of IL-17 by quantitative real-time PCR.

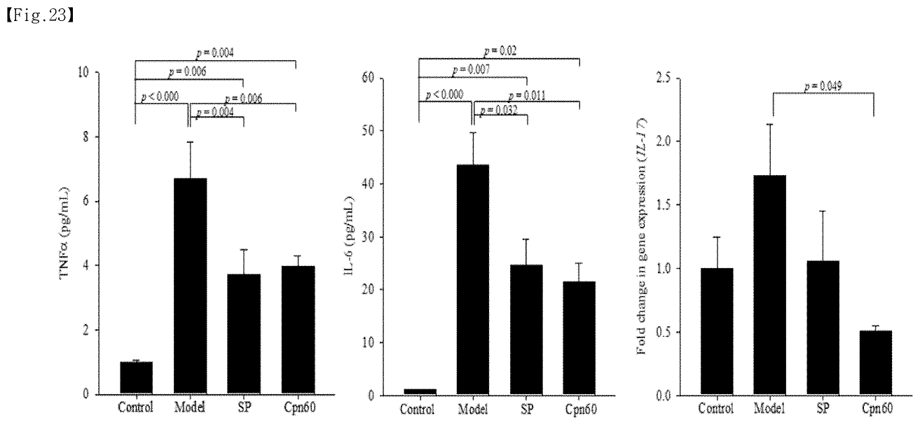

As shown in FIG. 23, levels of inflammatory cytokines TNFα, IL-6, and IL-17 were significantly decreased in the group administered the Cpn60 protein of the present disclosure compared with the arthritis-induced group.

These results confirmed that administration of the Cpn60 protein of the present disclosure can suppress excessive immune responses caused by rheumatoid arthritis and reduce levels of inflammatory cytokines.

### <11-6> Analysis of the Effect of 60 kDa Chaperonin Protein (Cpn60) Administration on Osteoclast Differentiation in Collagen-induced Rheumatoid Arthritis Mice

Synovium tissue was isolated from the mice used in Example 11-1, and the relative mRNA expression levels of factors related to osteoclast differentiation, including Nfatc1, Cask, and Calcr, were analyzed by quantitative real-time PCR.

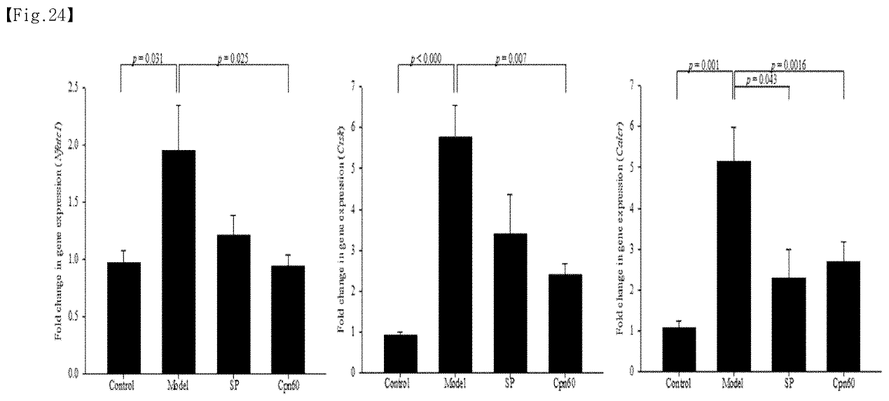

As shown in FIG. 24, expression levels of Nfatc1, Cask, and Calcr were significantly increased in the arthritis-induced group (model) compared with the control group, whereas in the group administered the Cpn60 protein of the present disclosure, expression levels of Nfatc1, Cask, and Calcr were significantly decreased.

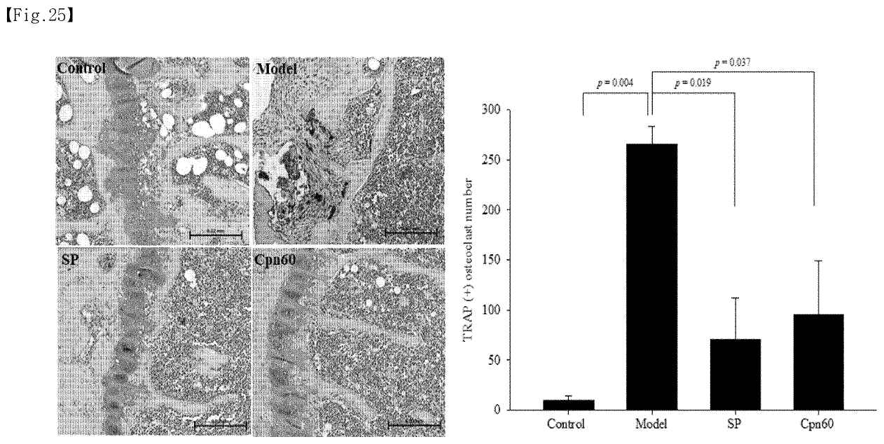

In addition, TRAP staining of the knee joint tissue showed the number that of TRAP-positive cells (osteoclasts) stained red-purple in the knee joint area was markedly increased in the arthritis-induced group compared with the control group. Meanwhile, in the group administered the Cpn60 protein of the present disclosure, the number of TRAP-positive cells was significantly decreased compared with the arthritis-induced group (see FIG. 25).

These results demonstrated that the extracellular vesicles and Cpn60 protein of the present disclosure isolated from Propionibacterium freudenreichii MJ2 strain can effectively inhibit osteoclast differentiation and improve inflammatory responses, thereby preventing, improving, and treating rheumatoid arthritis.

The present invention is the result of research conducted through the following national research and development project.
Project Number: 2022R1A2C1010264
Ministry: Ministry of Science and ICT
Managing Agency: National Research Foundation of Korea
Research Program: Mid-career Research Program (Type 1-1 (Integrated Type 1))
Research Task: Functional Characterization and Mechanistic Elucidation of Rheumatoid Arthritis Improvement by Chaperonin Derived from Propionibacterium
Contribution: 1/1
Research Institution: Korea University Research and Business Foundation
Research Period: 2022.03.01-2023.02.28

The preferred embodiments of the present disclosure have been described above. Those skilled in the art will appreciate that the present disclosure may be implemented in a modified form without departing from the essential characteristics of the present disclosure. Therefore, the disclosed embodiments should be considered illustrative rather than limiting. The scope of the present disclosure is defined by the appended claims, and all variations equivalent thereto are to be interpreted as being included within the scope of the present disclosure.

1. A method of preventing or treating rheumatoid arthritis in a subject in need thereof comprising: administering to the subject a pharmaceutically effective amount of a pharmaceutical composition comprising extracellular vesicles derived from Propionibacterium freudenreichii strain or 60 kDa chaperonin protein (Cpn60).

2. The method of claim 1, wherein the Propionibacterium freudenreichii is Propionibacterium freudenreichii MJ2 (Accession No.: KCCM12272P).

3. The method of claim 1, wherein the composition exhibits at least one activity selected from the group consisting of:
suppression of expression of osteoclast differentiation-related factors;
inhibition of production of inflammatory cytokines;
inhibition of TRAP (tartrate-resistant acid phosphatase) activity in osteoclasts;
increase of IL-10 expression;
alleviation of bone erosion; and
suppression of production of collagen antigen-specific IgG, IgG1, and IgG2a antibodies.

4. The method of claim 1, wherein the extracellular vesicles are contained in an amount of 1×10⁶ particles/mL to 1×10⁸ particles/mL in the composition.

5. The method of claim 1, wherein the extracellular vesicles have an average particle diameter of 150 to 200 nm.

6. A food composition for preventing or alleviating rheumatoid arthritis, the composition comprising extracellular vesicles derived from Propionibacterium freudenreichii strain or 60 kDa chaperonin protein (Cpn60) as an active ingredient.

7. The food composition of claim 6, wherein the Propionibacterium freudenreichii is Propionibacterium freudenreichii MJ2 (Accession No.: KCCM12272P).

8. The food composition of claim 6, wherein the composition exhibits at least one activity selected from the group consisting of:
suppression of expression of osteoclast differentiation-related factors;
inhibition of production of inflammatory cytokines;
inhibition of TRAP (tartrate-resistant acid phosphatase) activity in osteoclasts;
increase of IL-10 expression;
alleviation of bone erosion; and
suppression of production of collagen antigen-specific IgG, IgG1, and IgG2a antibodies.

9. The food composition of claim 6, wherein the extracellular vesicles are contained in an amount of 1×10⁶ particles/mL to 1×10⁸ particles/mL in the composition.

10. The food composition of claim 6, wherein the extracellular vesicles have an average particle diameter of 150 to 200 nm.
